(19) United States
(12) Patent Application Publication
(10) Pub. No.: US 2020/0407957 A1
(43) Pub. Date: Dec. 31, 2020

(54) ILLUMINATED SINK SYSTEM

(71) Applicant: Doug Vaught, Winter Garden, FL (US)

(72) Inventor: Doug Vaught, Winter Garden, FL (US)

(21) Appl. No.: 16/912,649

(22) Filed: Jun. 25, 2020

Related U.S. Application Data
(60) Provisional application No. 62/867,088, filed on Jun. 26, 2019.

(51) Int. Cl. E03C 1/186; E03C 1/126; A61L 2/10

(52) U.S. Cl. CPC E03C 1/186; A61L 2/10; E03C 1/126

(57) ABSTRACT

An illuminated sink system includes a sink basin having at least one sidewall, an upper rim, a base wall, a drain opening, and an interior space. A generally transparent body section is positioned along a portion of the sink basin, and a lighting unit is secured along the body section. The lighting unit including a plurality of lights for selectively illuminating the interior portion of the sink basin, and a plurality of ultraviolet lights for selectively sanitizing the interior portion of the sink basin. The system includes a controller that is connected to the lighting unit and a motion sensor.

### CROSS-REFERENCE TO RELATED APPLICATIONS

This application claims the benefit of U.S. Application Ser. No. 62/867,088 filed on Jun. 26, 2019, the contents of which are incorporated herein by reference.

### TECHNICAL FIELD

The present invention relates generally to bath and kitchen fixtures, and more particularly to an illuminated sink system.

### BACKGROUND

The statements in this section merely provide background information related to the present disclosure and may not constitute prior art.

Kitchen and bathroom sinks are found in virtually every building across the globe. Depending on the particulars of the location, the design of the sink itself may be purely functional, or the sink may include decorative designs and/or features. Additionally, some type of lighting is placed nearby to allow users to operate the sink. Although the light housing may include decorative elements, the light produced is intended to illuminate the room of which the sink is a small part.

In light of the above, it would be beneficial to provide an illuminated sink system that can function to provide light within the sink so as to provide the sink with increased functionality and additional aesthetic appeal. It would also be beneficial if one or more of the lights of the sink could provide ultraviolet light so as to sterilize the sink between uses.

### SUMMARY OF THE INVENTION

The present invention is directed to an illuminated sink system. One embodiment of the present invention can include a sink basin having at least one sidewall, an upper rim, a base wall, a drain opening and an interior space. A generally transparent body section can be positioned along a portion of the sink basin, and a lighting unit can be secured along the body section.

In one embodiment, the lighting unit can include a plurality of lights that are capable of illuminating the interior portion of the sink basin in any number of different colors, intensities and patterns. In one embodiment, the lighting unit can include a plurality of ultraviolet lights that are capable of sanitizing the interior portion of the sink basin.

In one embodiment, a controller is connected to the lighting unit and a motion sensor. The controller can function to selectively control the operation of the lighting unit based upon a timer output or a signal received from the motion sensor.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is an exploded parts view of the illuminated sink system, in accordance with one embodiment that is useful for understanding the inventive concepts disclosed herein.

FIG. 2 is a bottom perspective view of the illuminated sink system, in accordance with one embodiment of the invention.

FIG. 3 is a perspective view of the illuminated sink system, in accordance with one embodiment of the invention.

FIG. 4 is an exemplary flowchart of a system controller of the illuminated sink system, in accordance with one embodiment of the invention.

FIG. 5 is another perspective view of the illuminated sink system, in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting, but rather to provide an understandable description of the invention.

Although described for use in a kitchen or bathroom, this is for illustrative purposes only. To this end, the below sink system can be manufactured for use in any type of environment where a sink or water dispenser would be used. For example, other embodiments include surgical scrub sinks, drinking water fountains, and the like.

### Definitions

As described herein, a “unit” means a series of identified physical components which are linked together and/or function together to perform a specified function.

As described herein, the term “removably secured,” and derivatives thereof shall be used to describe a situation wherein two or more objects are joined together in a non-permanent manner so as to allow the same objects to be repeatedly joined and separated.

As described throughout this document, the term “complementary shape,” and “complementary dimension,” shall be used to describe a shape and size of a component that is identical to, or substantially identical to the shape and size of another identified component within a tolerance such as, for example, manufacturing tolerances, measurement tolerances or the like.

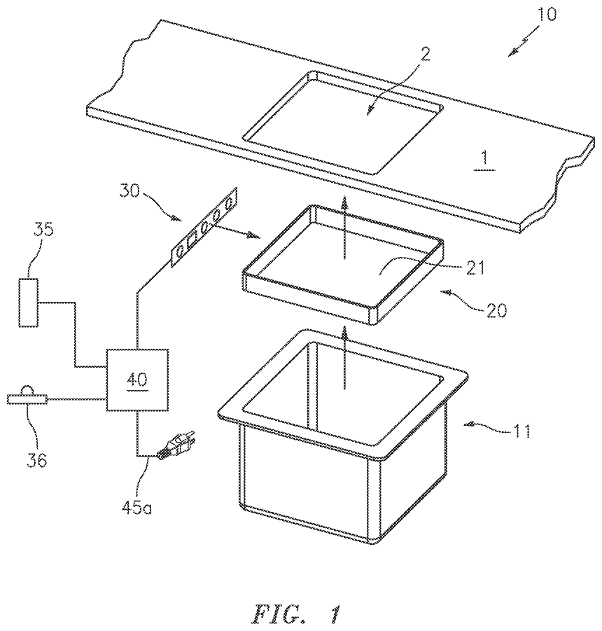

FIGS. 1-5 illustrate one embodiment of an illuminated sink system 10 that are useful for understanding the inventive concepts disclosed herein. In each of the drawings, identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 1.

As shown at FIG. 1, one embodiment of the illuminated sink system 10 can include, essentially, a sink basin 11, body section 20, a lighting unit 30 and a system controller 40. The system 10 can include or can be configured for use with any type of counter 1 having a sink opening 2 positioned therein.

As shown, the sink basin 11 can include one or more sidewalls 12, a base 13, and a drain opening 14 positioned therein. The sidewalls and base forming an interior surface for retaining water and washable items. In one embodiment, the top ends of the sidewalls can extend outward to form a continuous rim 15.

As described herein, the sink basin can include any number of different shapes and sizes and can be manufactured from any number of different materials such as stainless steel, porcelain, copper, or plastic, for example. Although described above, as including a single basin, this is for illustrative purposes only, as other embodiments are contemplated wherein the sink system 10 includes two or more individual sink basins 11.

In the preferred embodiment, the body section 20 can be constructed from a generally clear or translucent material such as acrylic, for example, having a size that is slightly (e.g., 1-3 inches) larger than the size of the basin rim 15. The body section can include a central opening 21 having a shape and size that is complementary to the shape and size of the top ends of the basin sidewalls 12.

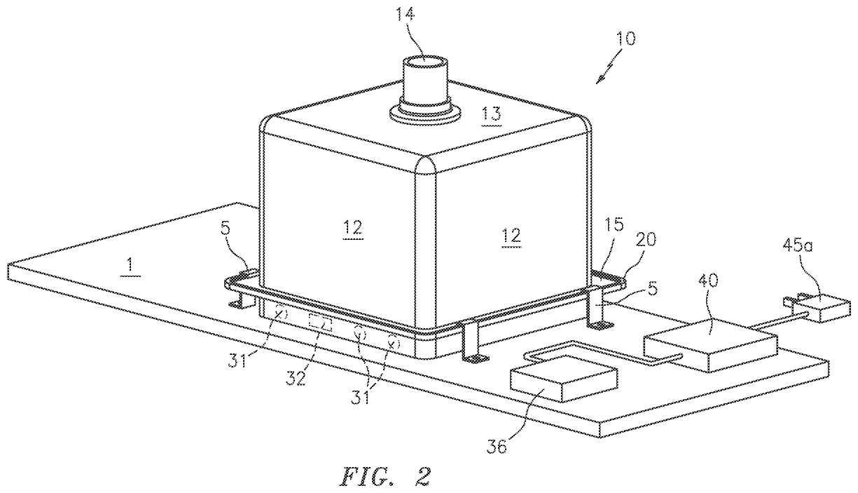

As shown, best at FIG. 2, the body section 20 can be positioned between the basin rim 15 and the bottom surface of the countertop 1 such that the counter opening 2, the body opening 21 and the basin base 13 are vertically aligned. When so positioned, any number of commercially available sink clips 5, or other such hardware can be used to secure the system to the counter.

Although described above with regard to particular materials, shapes and/or attachment components, this is for illustrated purposes only. To this end, the body section 20 can be constructed from any number of other materials and can include any number of shapes and sizes that are different from the rim portion 15.

Additionally, other embodiments are contemplated wherein the body section is permanently joined to the basin 11 via adhesives such as glue or resin, for example, and/or can be incorporated into the initial construction of the basin so as to form an integral component made from one or more materials. In such a situation, the insert may be positioned at locations along or within the basin 11 other than the rim.

The lighting unit 30 can be connected to the below described controller 40 and can function to disseminate one or more different types of light through the body section 20 and into the basin 11. In the preferred embodiment, the lighting unit can include a plurality of individual lights 31 such as Light Emitting Diodes (LED's), and/or LED strips, for example, that are capable of generating sustained visible light in a variety of different colors, patterns and intensities.

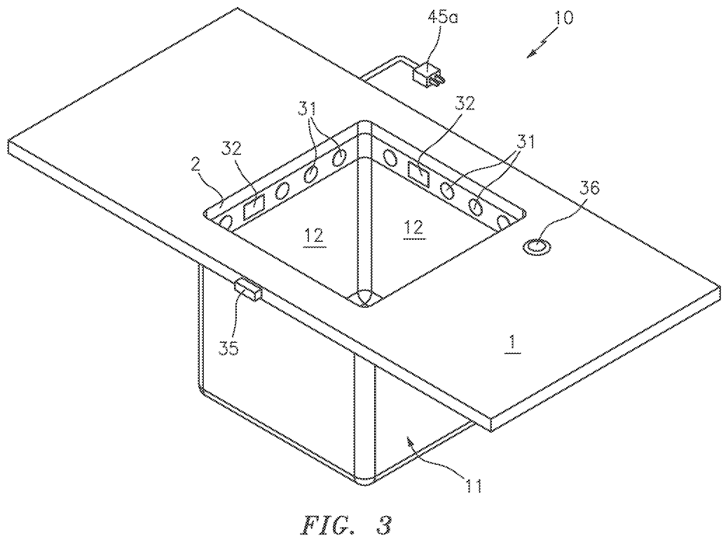

As shown best at FIG. 3, in the preferred embodiment, these lights 31 can be positioned along or within the entire body section 20 so as to illuminate the basin 11 from all sides. Of course, other embodiments are contemplated wherein the lights are positioned only along a portion of the body 20, and/or wherein other types of light producing devices are used.

In one embodiment, the lighting unit 30 can also include one or more ultraviolet lights 32 such as ultraviolet LED's, for example, that can also be provided along or within the body 20. The inclusion of ultraviolet lights can function to advantageously kill bacteria and certain viruses, thus adding a significant beneficial feature to the system when utilized in locations such as hospitals, nursing homes or medical facilities, for example.

In one embodiment, the system 10 can include a motion sensor 35 which can be placed so as to detect movement of a user nearby. The motion sensor can be communicatively linked to the system controller directly or wirelessly and can function to selectively activate and/or deactivate certain functionality. For example, upon detecting the motion of a user, the system can activate one or more of the lights 31, and can immediately deactivate the operation of the ultraviolet lights 32. Of course, other functionality can be tied to the sensor.

In one embodiment, the system 10 can include any number of user controls 36 such as the illustrated push button, for example. The user controls can be communicatively linked to the system controller 40 directly or wirelessly and can function to allow a user to control the operation of the lighting unit 30. To this end, the user controls can allow a user to turn the lights on or off, and/or to change the color, intensity, pattern or type of lights being utilized by the lighting unit. Of course, other functionality can be tied to the user controls.

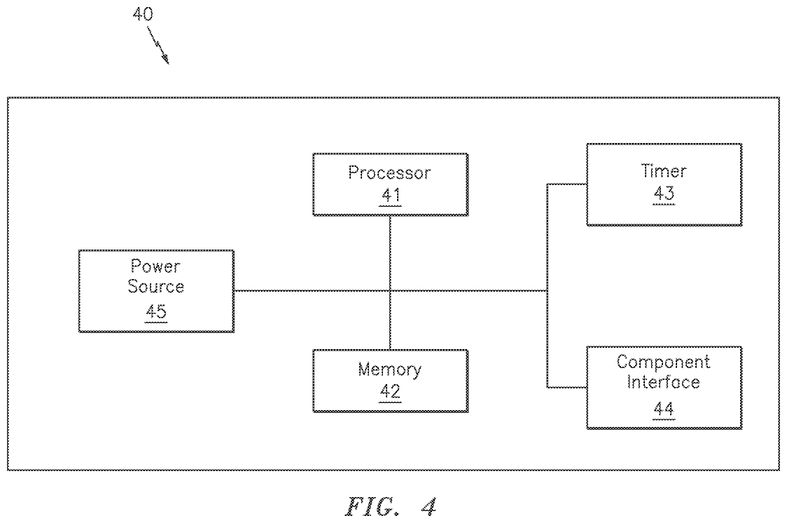

The controller 40 can control the use and operation of the system. As shown at FIG. 4, one embodiment of the controller can include a processor 41 that is communicatively linked to an internal memory 42, a timer module 43, a component interface unit 44, and/or a power source 45.

Although illustrated as separate elements, those of skill in the art will recognize that one or more system components may comprise, or include one or more printed circuit boards (PCB) containing any number of integrated circuit or circuits for completing the activities described herein. Of course, any number of other analog and/or digital components capable of performing the below described functionality can be provided in place of, or in conjunction with the below described controller elements.

The processing unit 41 can be a conventional central processing unit (CPU) or any other type of device, or multiple devices, capable of manipulating or processing information such as program code stored in the memory 42 and for causing the circuitry to complete the activities and functionality described herein.

Likewise, a timer module 43 can be provided, and can function to accurately measure the passage of time. As described herein, the timer module can be provided as a function of the processor or can include a separate physical circuit. In operation, the timer module can be used by the processor to selectively activate or deactivate the different lights 31 and 32 of the lighting units based on the passage of time.

Memory 42 can act to store operating instructions in the form of program code for the processor 41 to execute. Although illustrated in FIG. 4 as a single component, memory 42 can include one or more physical memory devices such as, for example, local memory and/or one or more bulk storage devices. As used herein, local memory can refer to random access memory or other non-persistent memory device(s) generally used during actual execution of program code, whereas a bulk storage device can be implemented as a persistent data storage device such as a hard drive, for example, containing programs that permit the processor to perform the functionality described below. Additionally, memory 42 can also include one or more cache memories that provide temporary storage of at least some program code in order to reduce the number of times program code must be retrieved from the bulk storage device during execution. Each of these devices are well known in the art.

The internal component interface unit 44 can function to provide a communicative link between the processor 41 and various other device components such as the lighting unit 30, the motion sensor 35 and/or the user controls 36. In this regard, the component interface unit can include any number of different components such as one or more PIC microcontrollers, internal bus, USB connections and other such hardware capable of providing a direct link between the various components. Of course, any other means for providing the two-way communication between the identified components can also be utilized herein.

In one embodiment, the power unit 45 can include an A/C electrical power transformer and cord 45a capable of allowing the device to be powered from a standard electrical outlet. Of course, other embodiments are also contemplated wherein the power source can include a plurality of batteries. Such a feature can be integrated into a smaller size main body so as to create a portable version of the device for use while traveling, for example.

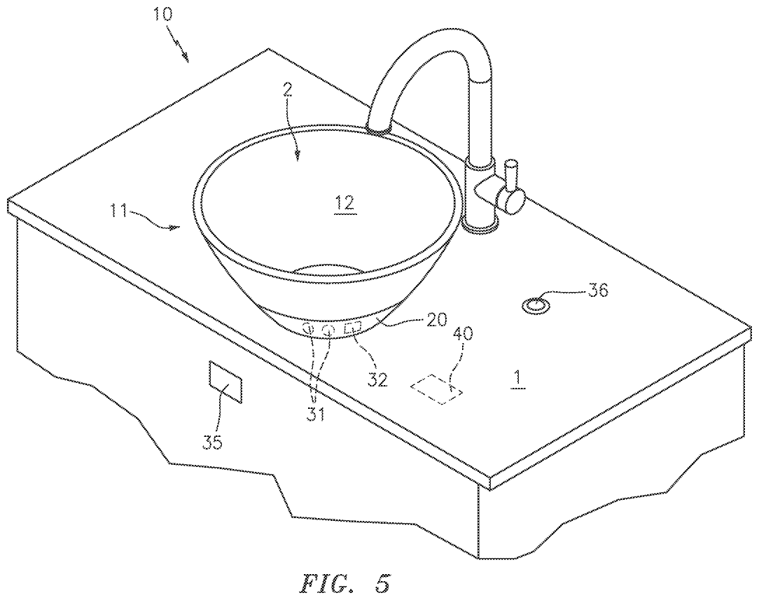

As noted above, the system components can be arranged at different locations so as to create or accommodate sink basins of varying shapes and sizes. To this end, FIG. 5 illustrates one embodiment of the system 10 wherein the body section 15 and lights 31 and/or 32 are positioned along the bottom end of the sink basin 11. Such a feature being particularly advantageous for the vase-style sink systems wherein the basin is not positioned beneath a countertop.

Although described above as a complete sink system, other embodiments are also contemplated wherein the body section 15, lighting unit 30 and/or controller 40, for example, are provided as an aftermarket upgrade for use with other sinks. In such an embodiment, the body section 15 can be constructed so as to be complementary to the shape and size of a portion of the sink basin to which the system is to be mated.

As described herein, one or more elements of the illuminated sink system 10 can be secured together utilizing any number of known attachment means such as, for example, screws, glue, compression fittings and welds, among others. Moreover, although the above embodiments have been described as including separate individual elements, the inventive concepts disclosed herein are not so limiting. To this end, one of skill in the art will recognize that one or more individually identified elements may be formed together as one or more continuous elements, either through manufacturing processes, such as welding, casting, or molding, or through the use of a singular piece of material milled or machined with the aforementioned components forming identifiable sections thereof.

As to a further description of the manner and use of the present invention, the same should be apparent from the above description. Accordingly, no further discussion relating to the manner of usage and operation will be provided.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Likewise, the terms “consisting” shall be used to describe only those components identified. In each instance where a device comprises certain elements, it will inherently consist of each of those identified elements as well.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

1. A sink system, comprising:
a sink basin having at least one sidewall, a base wall, and an interior space;
a lighting unit having a plurality of lights that are oriented toward the interior space of the sink basin; and
a controller that is in communication with the lighting unit, said controller functioning to control an operation of the lighting unit.

2. The system of claim 1, further comprising:
a body section that is connected to the sink basin, and
wherein the plurality of lights of the lighting unit are secured onto the body section.

3. The system of claim 2, wherein the body section is constructed from a transparent material.

4. The system of claim 3, wherein the plurality of lights of the lighting unit are positioned along an entirety of the body section.

5. The system of claim 1, further comprising:
a motion sensing device that is in communication with the controller.

6. The system of claim 5, wherein the controller includes functionality for selectively operating the lighting unit based upon an input from the motion sensing device.

7. The system of claim 2, further comprising:
a rim that is positioned along a top end of the at least one sidewall of the sink basin.

8. The system of claim 7, wherein the body section is configured to engage the rim, and
wherein the body section includes a central opening having a shape and a size that is complementary to a shape and a size of the top end of the at least one sidewall of the sink basin.

9. The system of claim 8, further comprising:
at least one sink clip that is configured to secure each of the rim and the body section to a counter.

10. The system of claim 1, wherein the controller further comprises:
a memory;
a power source;
a timer; and
a processor that is in communication with the memory, the power source, the timer and the lighting unit,
wherein the controller includes functionality for selectively operating the lighting unit based on an input from the timer.
